(19) United States
(12) Patent Application Publication
(10) Pub. No.: US 2026/0207714 A1
(43) Pub. Date: Jul. 23, 2026

(54) Methods for Bicameral, CNS+ Treatment of CNS, Cardiovascular, Metabolic and Other Disorders

(71) Applicant: Glenn Cornett, New York City, NY (US)

(72) Inventor: Glenn Cornett, New York City, NY (US)

(21) Appl. No.: 18/929,499

(22) Filed: Oct. 28, 2024

Related U.S. Application Data
(60) Provisional application No. 63/546,172, filed on Oct. 27, 2023.
(60) Provisional application No. 63/546,169, filed on Oct. 27, 2023.

(51) Int. Cl. A61K 38/30; A61K 9/00; A61K 31/4355; A61K 38/22; A61K 38/28

(52) U.S. Cl. CPC A61K 38/30; A61K 9/0043; A61K 31/4355; A61K 38/22; A61K 38/28

(57) ABSTRACT

Safe, effective, disease-modifying treatments—and especially, optimized treatments—for Alzheimer's and many other CNS, cardiovascular, metabolic and other disorders remain elusive. This is in part due to many CNS, cardiovascular, metabolic. and other disorders having underlying components on both sides of the blood-brain barrier—e.g., having CNS components as well as cardiovascular and/or metabolic and/or other non-CNS components. Respective examples in CNS, cardiovascular, metabolic and other domains include Alzheimer's disease, heart failure, diabetes and COVID-19. This is in and of itself a challenge, as most drugs (~98% of small molecules; a greater portion of larger molecules such as antibodies and peptides) do not cross the blood brain barrier appreciably, and even when they do, the chances of them having a partition coefficient that optimizes dosing in compartments on both sides of the blood brain barrier is vanishingly low. This invention addresses that challenge with a novel, bicameral, CNS+ (i.e., two-compartment, where one compartment is on the neuronal/glial side of the blood-brain barrier and one compartment is not) approach. This bicameral, CNS+ approach involves parallel
1) Intranasal, direct-to-brain delivery of drugs addressing the CNS component of a CNS, cardiovascular or metabolic disorder
2) Delivery (oral, injection or otherwise) of a cardiovascular &/or metabolic &/or other agent to the central compartment (i.e., blood and well-perfused organs [e.g., liver, kidneys]) to address cardiovascular &/or metabolic components &/or other components of a CNS, cardiovascular, metabolic or other disorder.

This allows for
1) Addressing more facets of complex CNS, cardiovascular, metabolic and other disorders
2) Focusing distribution of drugs on target areas so that more-effective dosages may be delivered.

### CLAIM OF PRIORITY TO EARLIER APPLICATION

This application is a Non-Provisional Patent Application and claims priority to and incorporates in its entirety U.S. Provisional Patent Application 63/546,172 filed on Oct. 27, 2023 and U.S. Provisional Patent Application 63/546,169 filed on Oct. 27, 2023.

### FIELD OF THE INVENTION

The present invention relates to methods and compositions for treating CNS, cardiovascular, metabolic and other disorders. The methods comprise administering at least two drugs, at least one to the brain (via intranasal, direct-to-brain delivery) and at least one to the central compartment (usually via, oral or injection routes).

At least one of the proposed systemic drugs (i.e., cicletanine) activates both (1) BKCs (large-conductance potassium channels) and (2) eNOS.

### DESCRIPTION OF FIGURES

To facilitate further description of the embodiments, the following drawings and descriptions, which should not be considered limiting in any way, are provided. The drawings do not illustrate every embodiment of the present invention. With reference to the accompanying drawings, like elements are numbered alike.

FIG. 1 illustrates a chart of a number of PubMed papers returned by search query: “neurovascular.” Query date: 23 Oct. 2023.

FIG. 2 illustrates a chart of a number of PubMed papers returned by search query: “Alzheimer's, neurovascular.” Query date: 22 Oct. 2023.

FIG. 3 illustrates a chart of a number of PubMed papers returned by search query: “Alzheimer's, endothelial.” Query date: 22 Oct. 2023.

FIG. 4. Illustrates aspect of activity of cicletanine (Drug A) at hBK channels (human BK channels; large-conductance potassium channels).

### BACKGROUND

As scientific understanding of human disease continues to evolve, disorders once considered to be caused principally by pathology in a single system (e.g., CNS) are increasingly understood in terms of pathologies critically driven by two or more systems (e.g., CNS+ cardiovascular).

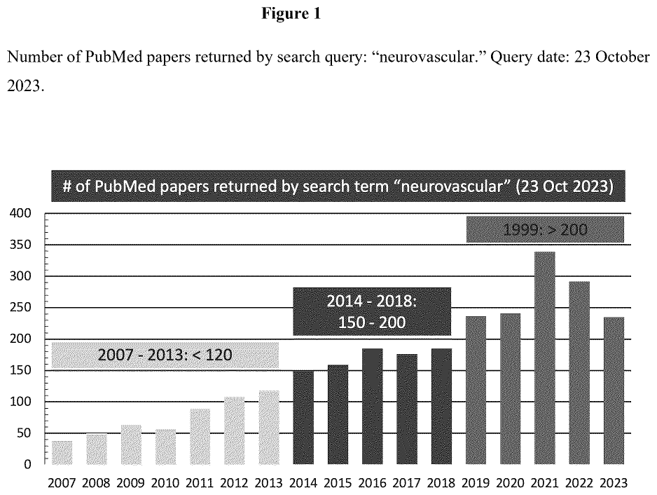

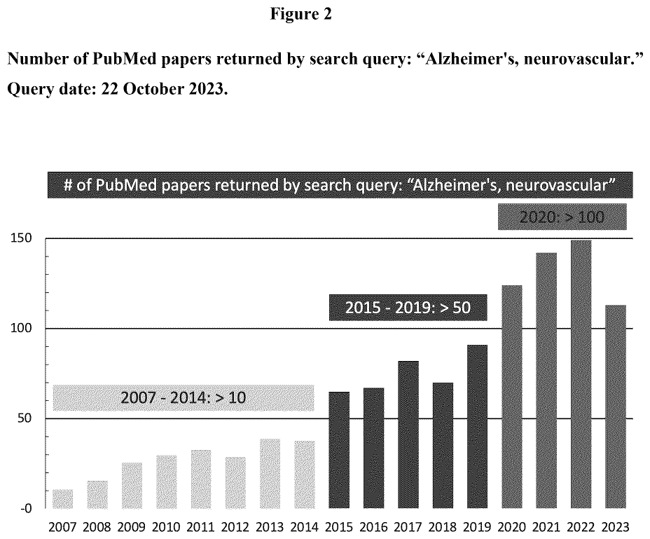

A relevant example here is Alzheimer's disease (AD). Originally characterized as driven by pathology mostly restricted to the CNS (central nervous system), AD is increasingly referred to as a pathology of both the brain and its associated vascular supply. This has helped spawn use of the term “neurovascular unit.” Canonically, the term “neurovascular unit” means a grouping of mutually-associated vascular cells, glial cells, and neurons that forms a minimal functional unit of the brain. Use of the term has continued to increase for the past couple decades. The per-year number of PubMed papers returned by the by search term “neurovascular” first exceeded 200 in 2019 and has since stayed above that despite the pandemic (FIG. 1). The neurovascular unit is a concept applied both to heathy and disordered brain states, though it has found particularly-concerted use in Alzheimer's disease. The per-year number of PubMed papers returned by the by search query “Alzheimer's, neurovascular” first exceeded 100 in 2020 and has since stayed above that threshold despite the pandemic (FIG. 2).

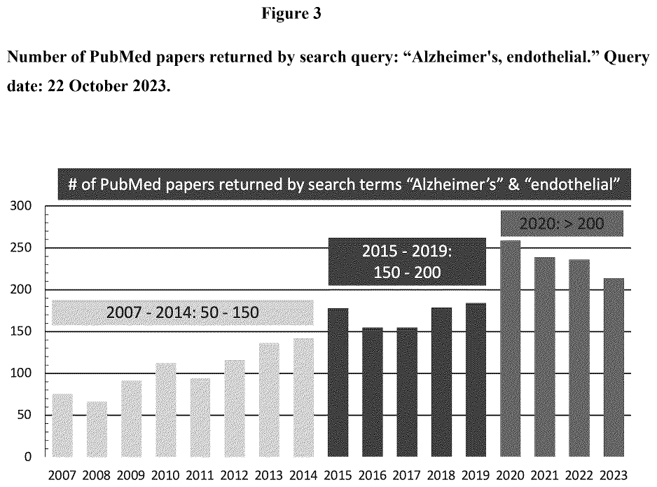

Critical to the understanding of the neurovascular pathology underlying Alzheimer's disease is the concept of endothelial dysfunction (EnD), which is caused by reduced activation of eNOS (endothelial nitric oxide synthase). This reduced eNOS activity (1) decreases the availability of nitric oxide (NO), an important regulator of blood pressure and coagulation and (2) increases the presence of reactive oxygen species (ROS) and reactive nitrogen species (RNS). These imbalances caused by EnD in turn are acknowledged as a critical driver of several critical cardiovascular and metabolic disorders, including hypertension, heart failure, angina, insulin resistance and vascular complications of diabetes. The importance of endothelial function is now also getting increased attention in the study of a range of pathologies, including CNS disorders, particularly those driven by dysfunction of the neurovascular unit. Alzheimer's again provides an excellent example. The per-year number of PubMed papers returned by the by search query “Alzheimer's, endothelial” first exceeded 200 in 2020 and has since stayed above that despite the pandemic (FIG. 3).

Despite the now-acknowledged importance of endothelial function in AD, this has resulted in virtually zero activity targeting endothelial dysfunction in AD, particularly toward anything that could be developed and commercialized. An analysis (23 Oct. 2023) of clinical activity at clincaltrials.gov found 633 Alzheimer's clinical trials that were (1) interventional (as opposed to observational only) and (2) either ongoing or about to start recruiting patients. Of those 633 trials, only 6 were returned when the term “endothelial dysfunction” or the term “endothelial” was added. Of these six studies, none was likely to result in a product that would be approved as a drug and reach patients. None of the six studies was sponsored by industry. Specifically of the six studies:
a. Two were lifestyle/diet intervention studies that did not involve the administration of a therapeutic drug;
b. Three involved diet supplements;
c. One involved dietary supplementation with Equol (an isoflavan extracted from soy);
d. One involved dietary supplementation with probiotics;
e. One involved dietary supplementation with the amino acids glycine and N-acetylcysteine; and
f. One involved the administration of hyperbaric oxygen.

All of this underscores the lack of rigorous approach to treating endothelial dysfunction in Alzheimer's disease. Importantly, none of these planned or ongoing trials, nor any Alzheimer's/endothelial dysfunction trials involves bicameral (two-compartment) therapy with (1) at least one agent delivered directly to the brain (intranasally or otherwise) and (2) at least one agent delivered systemically to the central compartment or any other target organ/tissue/area outside the brain.

The above substantiates, in AD, the neurological disease currently receiving the greatest amount of research funding from private firms and government agencies, the promise associated with and the novelty of the bicameral-therapy approach described in this invention.

The novelty and promise of bicameral therapy applies more broadly, both to
(1) other diseases classically thought to be CNS diseases (e.g., Parkinson's disease, and
(2) diseases classically associated principally with systems other than the CNS, including cardiovascular diseases, metabolic diseases and other disorders (e.g., COVID-19).

Examples of other CNS disorders where bicameral therapy is both novel and promising include Parkinson's disease, traumatic brain injury and vascular dementia.

Vascular dementia example: A clinical trials.gov search for interventional (i.e., not just observational) vascular dementia trials that are returned when the search term “Endothelial Dysfunction” is added yields six studies. Three of these were with diet supplements, two used lifestyle/diet modifications and one involved hyperbaric oxygen. None of them involved a bicameral approach that included direct-to-brain dosing.

Examples of “non-CNS” diseases where bicameral therapy in novel and promising include
Cardiovascular disease: Hypertension, heart failure, angina;
Metabolic disorders: Insulin resistance, diabetes, diabetes complications, obesity;
Other disorders: COVID-19 provides an excellent example of a disorder usually categorized outside of the CNS/cardiovascular/metabolic realm (in this case, an infectious disease), where bicameral therapy would be both novel and promising. On the one hand, Covid is now acknowledged as having critical endothelial-dysfunction components. On the other hand, the cognitive symptoms (e.g., “brain fog”) can be a significant factor, particularly as part of post-Covid syndrome (PCS; also called “long Covid”). Bicameral therapy, addressing both endothelial function (with a systemic endothelial-function agent, e.g., an eNOS activator) and cognitive function (with direct-to-brain administration of a neuroactive drug—e.g., a neuropeptide or another CNS-active peptide such as insulin) holds promise as an effective way to address this complex disease from two directions.

Also critical of the neurovascular pathology underlying Alzheimer's disease and other disorders addressed in this patent is the impairment of BKCs.

BKC impairment has been shown to be involved in the pathology of Alzheimer's disease, vascular dementia, heart failure, hypertension, diabetes and liver fibrosis (which in turn drives much of the pathology of cirrhosis in NASH, NAFLD, HCV cirrhosis and alcohol-related cirrhosis).

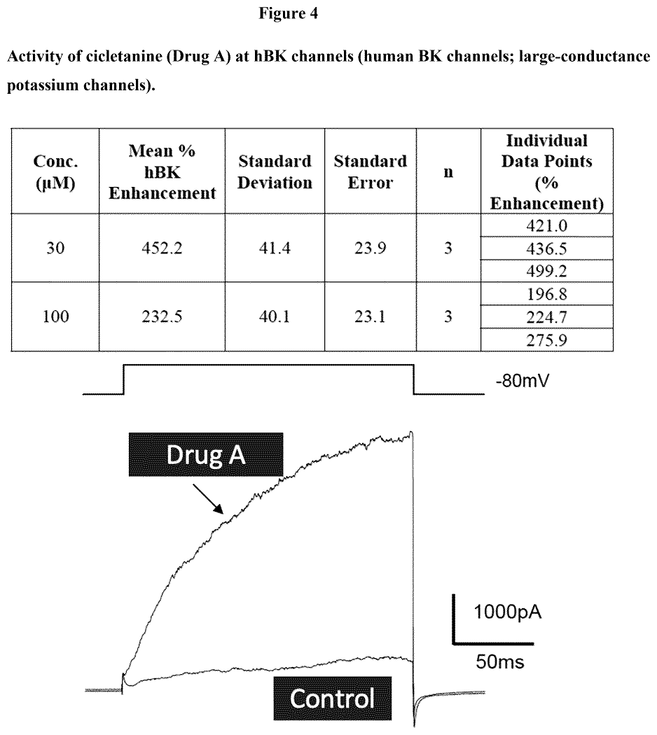

The BKC activity of cicletanine (FIG. 4) has been discovered to be particularly relevant and useful to the present inventions.

### BACKGROUND

A method of treating CNS, cardiovascular, metabolic, or other diseases with bicameral therapy is described here.

In its broadest description, “bicameral therapy” could refer to treatment of a disease discrete targeting of two different compartments (e.g., (1) brain, (2) central compartment), each with at least one drug.

Theoretically, bicameral therapy could involve delivery of the same drug to two different compartments, thereby being able deliver optimal dose to both compartments. For instance, one could, in a diabetic patient using insulin for blood-sugar control, augment that treatment with intranasal, direct-to-brain delivery of insulin to protect against cognitive aging. In most trials so far, especially for repeat dosing, therapeutic levels of insulin have been 10, 20 or 40 IU (as often as 4×/day). Acute dosing has gone at least as high as 160 IU. These levels are much higher than what could be safely delivered systemically. For example, usual mealtime doses of insulin are 0.2-0.4 IU/kg per day, divided into three doses. In a standard, 70 kg human, this works out to 14 to 28 units a day, dividing into 3×/daily doses ranging 4.8-9.6 IU/dose. Bicameral therapy therefore allows for delivering discretely and directly to the brain and make possible higher, more-beneficial concentrations of insulin in the brain than could be achieved by the systemic route (or be allowed in the systemic compartment). Conversely, discrete delivery of systemic insulin allows for therapeutically-viable amounts outside the brain.

In practice, while allowing for the possibility of delivering different levels of the same drug inside and outside the CNS, bicameral therapy is anticipated to be applied more frequently for the administration of (at least) two different drugs. An important example is that of an intranasal, direct-to-brain delivery of a neuro-active peptide (e.g., insulin, which (1) inhibits EF2k and GSK and (2) increases BDNF and important synaptic proteins such as synapsin and synaptophysin) along with oral delivery of a systemic agent for improving endothelial function (e.g., cicletanine, which directly corrects endothelial dysfunction by activating eNOS).

### DEFINITION OF TERMS

Prior to describing the invention in detail, it may be helpful to provide an understanding of certain terms that will be used:

“Bicameral, CNS+ therapy” refers to an approach or methodology involving delivery of drugs to two compartments, where one compartment is on the neuronal/glial side of the blood-brain barrier and one compartment is not.

“Bicameral therapy,” when used in this document, refers to “Bicameral, CNS+ therapy.”

The “central compartment” is the blood and well-perfused organs (e.g., liver, kidneys), to which drug distribution is virtually instantaneous once a drug is absorbed to the bloodstream.

Postoperative delirium (POD) is the development of delirium within the first 7 days after surgery under general anesthesia.

Delayed neurocognitive recovery (DNR) is the development of cognitive decline that occurs or persists within the first 30 days (but after the first 7 days) after surgery under general anesthesia.

Postoperative neurocognitive disorder (pNCD) is the development of cognitive decline that occurs or persists within the first year (but after the first 30 days) after surgery under general anesthesia.

In this document, the term “bicameral” refers specifically to bicameral, CNS+ approaches and methods.

### DETAILED DESCRIPTION

Herein is disclosed a method of bicameral, CNS+ therapy for the treatment of CNS, cardiovascular, metabolic and other diseases. Bicameral therapy involves delivery of drugs to (at least) two compartments, one drug targeting the brain (i.e. the neuronal/glial side of the blood-brain barrier), the other (usually but not necessarily) targeting the central compartment. An effective way to realize this is via (1) intranasal, direct-to-brain drug delivery and (2) systemic (oral, injection, etc.) delivery of a drug to the central compartment (i.e., blood and well-perfused organs [e.g., liver, kidneys]).

A bicameral approach can also be, as described below, applied to reversing the BKC hypoactivity that drives certain women's health disorders, including (among others) menopausal cognitive aging and menopausal sexual aging, by treatment with a BKC-activating agent. In some embodiments of the present invention, certain women's health disorders are addressed by treatment with an agent that activates both eNOS (endothelial nitric oxide synthase) and BKCs (BK channels; large-conductance potassium channels).

There is further disclosed the use of non-racemic cicletanine product either favoring the (−)cicletanine isomer or composed purely of the (−)cicletanine isomer. This allows for reduction or elimination of the diuretic effects of cicletanine, which are predominantly associated with the (+)cicletanine isomer. The benefits of this approach can be particularly important in situation where diuretic effects are particularly undesirable, such as BID dosing, where afternoon or evening dosing will likely have some diuretic effects persisting into sleeping hours, increasing the possibility of waking and rising from bed during the night in order to urinate.

FIG. 4 shows a chart representing aspects of cicletanine's BKC activity.

In embodiments herein, improvements over existing modes of therapy may be realized via parallel administration of agents (usually different ones) to the brain and a compartment outside of the brain.

In some embodiments, the diagnosed disorder or disorder being avoided is generally considered to be a CNS disorder, such as postoperative delirium, Alzheimer's disease, Parkinson's disease, multiple system atrophy, frontal temporal dementia, vascular dementia and so forth.

In one embodiment, a patient about to undergo surgery administers (1) 40 IU (international units) of insulin intranasally four times per day starting two days prior to surgery, continuing until discharge; (2) 150 mg cicletanine orally twice per day upon discharge from hospital, continuing to dose cicletanine for one week. As a result, the patient's experience of postoperative delirium (POD), &/or delayed neurocognitive recovery (DNR) &/or postoperative neurocognitive disorder (pNCD) is either diminished or completely avoided.

In one embodiment, a patient about to undergo surgery administers (1) 40 IU (international units) of insulin intranasally four times per day starting two days prior to surgery, continuing until discharge; (2) 150 mg cicletanine orally twice per day upon discharge from hospital, continuing to dose cicletanine for one month. As a result, the patient's experience of postoperative delirium (POD), &/or delayed neurocognitive recovery (DNR) &/or postoperative neurocognitive disorder (pNCD) is either diminished or completely avoided.

In one embodiment, a patient about to undergo surgery administers (1) 40 IU (international units) of insulin intranasally four times per day starting two days prior to surgery, continuing until discharge; (2) 150 mg cicletanine orally twice per day upon discharge form hospital, continuing to dose cicletanine for one year. As a result, the patient's experience of postoperative delirium (POD), &/or delayed neurocognitive recovery (DNR) &/or postoperative neurocognitive disorder (pNCD) is either diminished or completely avoided.

In one embodiment, a patient diagnosed with amnestic, mild cognitive impairment (aMCI; usually an early presentation of Alzheimer's disease) administers (1) 40 IU (international units) of insulin intranasally three times per day; (2) 100 mg cicletanine orally twice per day. As a result, the development of the patient's disorder is slowed, stopped or reversed.

In one embodiment, a patient diagnosed with Alzheimer's disease administers (1) 40 IU (international units) of insulin intranasally four times per day; (2) 150 mg cicletanine orally twice per day. As a result, the development of the patient's disorder is slowed, stopped or reversed.

In one embodiment, a patient diagnosed with Parkinson's disease administers (1) 1 g of gamma globulin intranasally divided evenly across 20 doses given over the course of 5 days (2) 150 mg cicletanine orally twice per day. As a result, the development of the patient's disorder is slowed, stopped or reversed.

In one embodiment, a patient diagnosed with multiple system atrophy (MSA) administers (1) 1 g of gamma globulin intranasally divided evenly across 20 doses given over the course of 5 days (2) 150 mg cicletanine orally twice per day. As a result, the development of the patient's disorder is slowed, stopped or reversed.

In one embodiment, a patient diagnosed with amnestic, mild cognitive impairment (aMCI; usually an early presentation of Alzheimer's disease) administers (1) 40 IU (international units) of an insulin analogue intranasally three times per day; (2) 100 mg cicletanine orally twice per day. As a result, the development of the patient's disorder is slowed, stopped or reversed.

In one embodiment, a patient diagnosed with Alzheimer's disease administers (1) 20 IU (international units) of an insulin analogue intranasally four times per day; (2) 150 mg cicletanine orally twice per day. As a result, the development of the patient's disorder is slowed, stopped or reversed.

In one embodiment, a patient diagnosed with Alzheimer's disease administers (1) 20 IU (international units) of IGF-1 intranasally four times per day; (2) 150 mg cicletanine orally twice per day. As a result, the development of the patient's disorder is slowed, stopped or reversed.

In one embodiment, a patient diagnosed with Alzheimer's disease administers (1) 20 IU (international units) of an IGF-1 analogue intranasally four times per day; (2) 150 mg cicletanine orally twice per day. As a result, the development of the patient's disorder is slowed, stopped or reversed.

In one embodiment, a patient diagnosed with frontotemporal dementia (FTD) administers (1) 20 IU (international units) of an insulin analogue intranasally four times per day; (2) 150 mg cicletanine orally twice per day. As a result, the development of the patient's disorder is slowed, stopped or reversed.

In one embodiment, a patient diagnosed with depression administers (1) 20 IU (international units) of an insulin analogue intranasally four times per day; (2) 150 mg cicletanine orally twice per day. As a result, the development of the patient's disorder is slowed, stopped or reversed.

In one embodiment, a patient diagnosed with bipolar disorder administers (1) 20 IU (international units) of an insulin analogue intranasally three times per day; (2) 150 mg cicletanine orally twice per day. As a result, the development of the patient's disorder is slowed, stopped or reversed.

In one embodiment, a patient diagnosed with attention-deficit/hyperactivity disorder (ADHD) administers (1) 20 IU (international units) of an insulin analogue intranasally two times per day; (2) 100 mg cicletanine orally twice per day. As a result, the development of the patient's disorder is slowed, stopped or reversed.

In one embodiment, a patient diagnosed with attention-deficit disorder (ADD) administers (1) 30 IU (international units) of an insulin analogue intranasally two times per day; (2) 100 mg cicletanine orally twice per day. As a result, the development of the patient's disorder is slowed, stopped or reversed.

In one embodiment, a patient diagnosed with borderline personality disorder administers (1) 20 IU (international units) of insulin intranasally four times per day; (2) 100 mg cicletanine orally twice per day. As a result, the development of the patient's disorder is slowed, stopped or reversed.

In some embodiments, the diagnosed disorder or disorder being avoided is generally not considered to be a CNS disorder, what is instead principally considered to be a cardiovascular, metabolic or other disorder.

In some embodiments, the diagnosed disorder or disorder being avoided is principally considered to be a cardiovascular disorder. Example disorders include but are not limited to heart failure, hypertension and angina.

In one embodiment, a patient diagnosed with heart failure administers (1) 20 IU (international units) of an insulin analogue intranasally four times per day; (2) 200 mg cicletanine orally twice per day. As a result, the development of the patient's disorder is slowed, stopped or reversed.

In one embodiment, a patient diagnosed with hypertension administers (1) 20 IU (international units) of insulin intranasally four times per day; (2) 150 mg cicletanine orally twice per day. As a result, the development of the patient's disorder is slowed, stopped or reversed.

In one embodiment, a patient diagnosed with angina administers (1) 20 IU (international units) of an insulin analogue intranasally four times per day; (2) 200 mg cicletanine orally twice per day. As a result, the development of the patient's disorder is slowed, stopped or reversed.

In some embodiments, the diagnosed disorder or disorder being avoided is principally considered to be a metabolic disorder or cardiometabolic disorder. Example disorders include but are not limited to diabetes, insulin resistance, high cholesterol, high triglycerides, metabolic syndrome, NAFLD (non-alcoholic fatty liver disease) or NASH (non-alcoholic steatohepatitis).

In one embodiment, a patient diagnosed with Type 2 diabetes administers (1) 30 IU (international units) of an insulin analogue intranasally four times per day; (2) 150 mg cicletanine orally twice per day. As a result, the development of the patient's disorder is slowed, stopped or reversed. Insulin sensitivity is increased. HbA1c is decreased. Microvascular complications of diabetes are ameliorated or avoided.

In one embodiment, a patient diagnosed with insulin resistance administers (1) 20 IU (international units) of an insulin analogue intranasally four times per day; (2) 200 mg cicletanine orally twice per day. As a result, the development of the patient's disorder is slowed, stopped or reversed.

In one embodiment, a patient diagnosed with high cholesterol administers (1) 20 IU (international units) of an insulin analogue intranasally four times per day; (2) 200 mg cicletanine orally twice per day. As a result, the development of the patient's disorder is slowed, stopped or reversed.

In one embodiment, a patient diagnosed with high triglycerides administers (1) 30 IU (international units) of an insulin analogue intranasally four times per day; (2) 100 mg cicletanine orally twice per day. As a result, the development of the patient's disorder is slowed, stopped or reversed.

In one embodiment, a patient diagnosed with metabolic syndrome administers (1) 20 IU (international units) of an insulin analogue intranasally four times per day; (2) 200 mg cicletanine orally twice per day. As a result, the development of the patient's disorder is slowed, stopped or reversed. Triglycerides and/or blood pressure and/or insulin resistance are decreased.

In one embodiment, a patient diagnosed NAFLD (non-alcoholic fatty liver disease) administers (1) 30 IU (international units) of an insulin analogue intranasally four times per day; (2) 150 mg cicletanine orally twice per day. As a result, the development of the patient's disorder is slowed, stopped or reversed.

In one embodiment, a patient diagnosed with NASH (non-alcoholic steatohepatitis) administers (1) 40 IU (international units) of an insulin analogue intranasally four times per day; (2) 150 mg cicletanine orally twice per day. As a result, the development of the patient's disorder is slowed, stopped or reversed.

In one embodiment, a patient diagnosed with NASH (non-alcoholic steatohepatitis) complicated by cirrhosis with ascites administers (1) 40 IU (international units) of an insulin analogue intranasally four times per day; (2) 250 mg cicletanine orally twice per day. As a result, the development of the patient's disorder is slowed, stopped or reversed. The patient's ascites is reduced, resulting in less fluid buildup and reduced need for large-volume paracentesis. The patient's hyperkalemia, brought on by spironolactone or other MRAs (Mineralocorticoid Receptor Antagonists) is reduced by means of the kaliuresis brought on by the higher-dose cicletanine.

In some embodiments, the diagnosed disorder or disorder being avoided is principally considered to be neither a CNS disorder, nor a cardiovascular disorder, nor a metabolic disorder. COVID-19 serves as a good example in this case.

In one embodiment, a patient diagnosed with COVID-19 administers (1) 40 IU (international units) of an insulin analogue intranasally four times per day; (2) 150 mg cicletanine orally twice per day. As a result, the development of the patient's disorder is slowed, stopped or reversed. One or more of the patient's cognition, olfaction, pulmonary function or exercise capacity benefit from the treatment.

Disorders presenting during perimenopause and postmenopause (hereafter referred to by the simplifying term, “menopause”) cause significant discomfort, morbidity and mortality. While it is standard practice to use cardiovascular agents to treat some aspects of menopause (e.g., hypertension, heart failure) it is not common to treat other aspects of menopause (e.g., hot flashes, sexual aging, cognitive aging) with a cardiovascular agent.

The idea of using cicletanine, a hypertension agent, for menopausal disorders such as hot flashes and sexual aging is both novel and unexpected. Cicletanine has been on the market in France and other countries since 1986. It has 210 PubMed papers. A general internet search and a PubMed query using the terms “cicletanine” and “menopause” returns no responses that suggest therapeutic relevance of cicletanine to menopausal disorders such as hot flashes and sexual aging.

Additionally, BKC (BK channel) activation is critical to the therapeutic activity described here. We have discovered that cicletanine has significant and beneficial BKC-activating properties (FIG. 4).

As used herein, “BKC” stands for large-conductance potassium (BK) channels.

As used herein, “menopause” encompasses both perimenopausal and postmenopausal phases of life.

A number of women's health disorders during menopause are driven by impaired activity of BK channels (BKCs; large-conductance potassium channels).

Menopausal vascular aging is known to be a causative factor in menopausal cognitive aging. Menopausal vascular aging has been shown to involve impairment of BKC function. Cicletanine activates BK channels (FIG. 1). This patent application (along with its associated patent application, Methods for Bicameral, CNS+ Treatment of CNS, Cardiovascular, Metabolic and Other Disorders) pursues patent protection for our discovery.

Menopausal OAB (overactive bladder) is associated with BKC impairment.

Menopausal vasomotor symptoms (“hot flashes”) are attributed in part to BK-channel activity (via decreased estrogen-beta receptor activity).

Menopausal sexual aging (e.g., vaginal atrophy, vaginal dryness) is also thought to be due in part BK channel impairment secondary to decreased estrogen levels.

Some embodiments of the present invention comprise the use cicletanine and other BKC agents as well as other agents, including intra-nasally administered agents, as set out below in the treatment of menopausal disorders, including but not limited to vascular aging, cognitive aging, sexual aging, hot flashes and overactive bladder.

This invention further discloses the use of non-racemic cicletanine product either favoring the (−)cicletanine isomer or composed purely of the (−)cicletanine isomer. This allows for reduction or elimination of the diuretic effects of cicletanine, which are predominantly associated with the (+)cicletanine isomer. The benefits of this approach can be particularly important in situation where diuretic effects are particularly undesirable, such as
1) overactive bladder, where enhanced diuretic activity is already a clinical problem;
2) cognitive impairment—particularly when the drug is dosed BID, thereby likely having some diuretic effects persisting into sleeping hours, increasing the possibility of waking and rising from bed during the night in order to urinate.
3) sexual aging—particularly when the drug is dosed in the afternoon or evening prior to intercourse, (or when the drug is dosed BID) thereby likely having some diuretic effects persisting into sleeping hours, increasing the possibility of waking and rising from bed during the night in order to urinate.

In one embodiment, female patients diagnosed with cognitive aging associated with amnestic mild cognitive impairment enter a placebo-controlled trial with cicletanine dosed orally 100 mg BID. At 6 months, the cicletanine patients are faring better than placebo patients, as measured by changes in ADAS-cog 12 (cognitive-performance measure) and/or a CogStat battery (cognitive-performance measure) and/or EQ-5D-5L (quality-of-life measure).

In one embodiment, female patients diagnosed with cognitive aging associated with Alzheimer's disease enter a placebo-controlled trial with cicletanine dosed orally 150 mg BID. At 12 months, the cicletanine patients are faring better than placebo patients, as measured by changes in ADAS-cog 12 (cognitive-performance measure) and/or a CogStat battery (cognitive-performance measure) and/or EQ-5D-5L (quality-of-life measure).

In one embodiment, female patients diagnosed with cognitive aging associated with Parkinson's disease enter a placebo-controlled trial with cicletanine dosed orally 150 mg BID. At 6 months, the cicletanine patients are faring better than placebo patients, as measured by changes in Unified Parkinson Disease Scale (UPDRS; cognitive, motor and general performance measures) and/or a CogStat battery (cognitive-performance measure) and/or EQ-5D-5L (quality-of-life measure).

In one embodiment, female patients diagnosed with sexual aging associated with menopause enter a placebo-controlled trial with cicletanine dosed orally 150 mg BID. At 6 months, the cicletanine patients are faring better than placebo patients, as measured by sexual function/satisfaction assessment and/or EQ-5D-5L (quality-of-life measure). Vaginal lubrication has been improved.

In one embodiment, female patients diagnosed with sexual aging associated with menopause enter a placebo-controlled trial with (−)cicletanine (isomer) dosed orally 100 mg BID. At 6 months, the cicletanine patients are faring better than placebo patients, as measured by sexual function/satisfaction assessment and/or EQ-5D-5L (quality-of-life measure). Vaginal lubrication has been improved.). The use of the (−)cicletanine isomer has allowed for elimination of the diuretic effect caused by the (+)cicletanine isomer.

In one embodiment, female patients diagnosed with vasomotor symptoms (“hot flashes”) associated with menopause enter a placebo-controlled trial with cicletanine dosed orally 150 mg BID. At 6 months, the cicletanine patients are faring better than placebo patients, as measured by an assessment of vasomotor symptoms and/or EQ-5D-5L (quality-of-life measure).

In one embodiment, female patients diagnosed with overactive bladder (OAB) associated with menopause enter a placebo-controlled trial with cicletanine dosed orally 150 mg BID. At 6 months, the cicletanine patients are faring better than placebo patients, as measured by an assessment of bladder function and/or EQ-5D-5L (quality-of-life measure).

In one embodiment, female patients diagnosed with overactive bladder (OAB) associated with menopause enter a placebo-controlled trial with (−)cicletanine (isomer) dosed orally 100 mg BID. At 6 months, the cicletanine patients are faring better than placebo patients, as measured by an assessment of bladder function and/or EQ-5D-5L (quality-of-life measure). The use of the (−)cicletanine isomer has allowed for elimination of the diuretic effect caused by the (+)cicletanine isomer.

In one embodiment, hospitalized female patients >65 years old enter a placebo-controlled trial with cicletanine dosed orally 150 mg BID. At 1 week, 1 month, and 12 months, the cicletanine patients have fared better than placebo patients in terms of (a) incidence of delirium and/or (b) per-patient days of delirium and/or (c) a CogStat battery (cognitive-performance measure) and/or (d) EQ-5D-5L (quality-of-life measure).

In one embodiment, hospitalized female patients >65 years old and in an intensive care unit enter a placebo-controlled trial with extended release cicletanine dosed orally 300 mg QD. At 1 week, 1 month, and 12 months, the cicletanine patients have fared better than placebo patients in terms of (a) incidence of delirium and/or (b) per-patient days of delirium and/or (c) a CogStat battery (cognitive-performance measure) and/or (d) EQ-5D-5L (quality-of-life measure).

In one embodiment, a female patient with orgasmic dysfunction administers (1) 20 IU (international units) of oxytocin 30 minutes prior to engaging in intercourse; (2) 150 mg 5:1 (31 ):(+)cicletanine (125 mg (−)cicletanine+25 mg (+)cicletanine) orally twice per day. As a result, the patient is able to reach orgasm.

In one embodiment, a male patient with orgasmic dysfunction administers (1) 30 IU (international units) of oxytocin 5 to 60 minutes prior to engaging in intercourse; (2) 125 mg 4:1 (−):(+)cicletanine (100 mg (−)cicletanine+25 mg (+)cicletanine) orally twice per day. As a result, the patient is able to reach orgasm and has a decrease in both relative and absolute refractory periods.

In one embodiment, a female patient with sexual arousal disorder administers (1) 10 IU (international units) of oxytocin 5 to 90 minutes prior to engaging in intercourse; (2) 125 mg (−)cicletanine orally twice per day. As a result, the patient is able to be aroused sexually; vaginal lubrication is increased, and sexual intercourse is more pleasureable.

In one embodiment, a female patient with GSM (genitourinary syndrome of menopause) administers (1) 30 IU (international units) of oxytocin 5 to 90 minutes prior to engaging in intercourse; (2) 50 mg (−)cicletanine orally twice per day. As a result, vaginal lubrication is increased, and sexual intercourse becomes pleasurable rather than painful.

In one embodiment, female patients diagnosed with vasomotor symptoms of menopause (VSM, or “hot flashes”) enter a placebo-controlled trial with (−)cicletanine dosed orally 100 mg BID and an intranasal insulin analogue at 20 IU (international units) twice daily. At 6 months, the [cicletanine+intranasal insulin analogue] patients are faring better than placebo patients, as measured by an assessment of vasomotor symptoms (assessed by Gynogram or other relevant means) and/or EQ-5D-5L (quality-of-life measure).

In one embodiment, a female patient diagnosed with overactive bladder (OAB) administers (1) 30 IU (international units) of intranasal insulin twice daily; (2) 125 mg 4:1 (−):(+)cicletanine (100 mg (−)cicletanine+25 mg (+)cicletanine) orally twice per day. At 6 months, the patient's bladder function and quality of life are improved significantly.

In one embodiment, hospitalized patients >65 years old and in an intensive care unit enter a placebo-controlled trial with (1) extended release cicletanine dosed orally 300 mg QD and (2) and intranasal insulin analogue dosed at 40 international units (IU) four times daily. At 1 week, 1 month, and 12 months, the [cicletanine+intranasal insulin analogue] patients have fared better than placebo patients in terms of (a) incidence of delirium and/or (b) per-patient days of delirium and/or (c) a CogStat battery (cognitive-performance measure) and/or (d) EQ-5D-5L (quality-of-life measure).

Although the invention has been described with reference to specific embodiments, it will be understood by those skilled in the art that various changes can be made without departing from the spirit or scope of the invention. Accordingly, the disclosure of embodiments is intended to be illustrative of the scope of the invention and is not intended to be limiting. It is intended that the scope of the invention shall be limited only to the extent required by the appended claims. To one of ordinary skill in the art, it will be readily apparent that the systems and methods discussed herein may be implemented in a variety of embodiments, and that the foregoing discussion of certain of these embodiments does not necessarily represent a complete description of all possible embodiments. Rather, the detailed description of the drawings, and the drawings themselves, disclose at least one preferred embodiment, and may disclose alternative embodiments.

What is claimed is:

1. The use of bicameral, CNS+ therapy for prevention and treatment of human disorders, where (a) one agent is administered by intranasal, direct-to-brain delivery and (b) a second agent is delivered systemically via oral, injection, transdermal, transmucosal or other routes.

2. The method of claim 1, where the intranasal agent administered is a neuroactive peptide.

3. The method of claim 1, where the intranasal agent administered is an antibody.

4. The method of claim 1, where the intranasal agent administered is neither an antibody nor a neuroactive peptide.

5. The method of claim 2, where the intranasal agent administered is either IGF-1, an IGF-1 analogue, an orexin, an orexin analogue, an insulin or an insulin analogue.

6. The method of claim 1, where the second agent enhances endothelial function and BKC activity.

7. The method of claim 6, where the second agent that enhances endothelial function is a furopyridine product.

8. The method of claim 7, where the furopyridine product is cicletanine (racemic or isomer-enhanced) or a cicletanine analogue (racemic or isomer-enhanced).

9. The method of claim 1, where the human disorder treated is a CNS disorder.

10. The method of claim 9, where the CNS disorder is postoperative delirium (POD), &/or delayed neurocognitive recovery (DNR) &/or postoperative neurocognitive disorder (pNCD).

11. The method of claim 9, where the CNS disorder is Alzheimer's disease, vascular dementia or another form of dementia, depression, bipolar disorder, ADD or ADHD.

12. The method of claim 1, where the human disorder treated is a genitourinary disorder or sexual-function disorder.

13. The method of claim 2, where the human disorder treated is a genitourinary disorder or sexual-function disorder.

14. The method of claim 1, where the disorder is a cardiovascular disorder.

15. The method of claim 14, where the disorder is heart failure, angina or pulmonary hypertension.

16. The method of claim 1, where the disorder is a metabolic or cardiometabolic disorder.

17. The method of claim 2, where the human disorder treated is a CNS disorder.

18. The method of claim 2, where the disorder is a cardiovascular disorder.

19. The method of claim 22, where the disorder is heart failure, angina or pulmonary hypertension.

20. The method of claim 1, where the human disorder treated is COVID-19.
